(19) United States
(12) Patent Application Publication
(10) Pub. No.: US 2008/0044821 A1
(43) Pub. Date: Feb. 21, 2008

(54) NUCLEIC ACID ARRAY HAVING FIXED NUCLEIC ACID ANTI-PROBES AND COMPLEMENTARY FREE NUCLEIC ACID PROBES

(76) Inventors: Gafur Zainiev, West Bloomfield, MI (US); Inlik Zainiev, West Bloomfield, MI (US)

Correspondence Address: GIFFORD, KRASS, SPRINKLE,ANDERSON & CITKOWSKI, P.C, PO BOX 7021, TROY, MI, 48007-7021, US

(21) Appl. No.: 11/465,870

(22) Filed: Aug. 21, 2006

(51) Int. Cl. C12Q 1/68; C07H 21/04

(52) U.S. Cl. 435/6; 536/24.3

(57) ABSTRACT

A process for identifying a complementary nucleic acid probe to a target nucleic acid involves forming an array of spots where each spot of the array has an immobilized nucleic acid anti-probe to which is hybridized a nucleic acid probe. The array of the anti-probe-probe complex is denatured. The nucleic acid probes are then moved into a target chamber that includes a target nucleic acid. Hybridization conditions are established to form double-stranded complexation between the target nucleic acid and nucleic acid probes in instances where the target nucleic acid has a sequence complementary. The nucleic acid probes noncomplementary to the target nucleic acid are allowed to rehybridize with anti-probes. Determining whether the anti-probe spots exposed to nucleic acid probes noncomplementary to the target nucleic acid are single stranded after exposure to noncomplementary nucleic acid probes provides information as to target nucleic acid sequence.

### FIELD OF THE INVENTION

The present invention in general relates to nucleic acid arrays, and in particular to the use of immobilized anti-probe nucleic acids to facilitate detection.

### BACKGROUND OF THE INVENTION

A DNA microarray (DNA chip) can be defined as a high-density array of short DNA molecules bound to a solid surface for use in probing a biological sample to determine gene expressions and nucleotide sequence of DNA and/or RNA.

Another definition could be that a DNA chip is a microchip that holds DNA probes that form half of the DNA double helix and can recognize DNA from samples being tested by hybridizing with another half of said DNA double helix.

The principle of DNA microarray technology is based on the fact that complementary sequences of DNA can be used to hybridize immobilized DNA molecules, where hybridization is the process of joining two complementary strands of DNA to form a double-stranded molecule. Ideally, each single-stranded molecule of DNA will only bind to its appropriate complementary target sequence on the immobilized array.

Typical for operating all kinds of DNA microarrays (chips) is hybridization of long DNA target molecules directly on the surfaces of DNA chip with short oligonucleotides tethered to the surface.

In the literature there exist at least two confusing nomenclature systems for referring to hybridization partners. Both use common terms: “probes” and “targets”. According to the nomenclature recommended by B. Phimister of Nature Genetics, a “probe” is the tethered nucleic acid with known sequence, whereas a “target” is the free nucleic acid sample whose identity/abundance is being detected. This patent specification follows the Phimister recommended nomenclature. See Nature Genetics volume 21 supplement pp. 1-60, 1999.

At the same time it is well recognized and accepted by those skilled in the art that short DNA targets are better able than large targets to interact with tethered oligonucleotides: they are less likely to have bases hidden from duplex formation by intramolecular base pairing; and, as they are less bulky, they will more readily penetrate the closely packed lawn of oligonucleotides. Ideally, target and probe should have the same length. (Nature Genetics 1999, 21, 5-9; BioTechniques. 2005, 39, 89-96).

The instant invention suggests performing DNA diagnostics in a way that does not require hybridization of long DNA target molecules directly on the DNA chip.

The oldest type of DNA microarray is the sequencing chip. This is also the type most commonly discussed in popular articles about this technology. With sequencing chips, such as those initially produced by Affymetrix or Hyseq, segments of DNA (usually 20 bases long) are placed in a microarray. Target samples are then introduced to the chip and the segment that the sample hybridizes with determines the result.

The second variety of DNA microarrays is the expression chip. These are designed to determine the degree of expression of a certain genetic sequence by measuring the rate or amount of messenger ribonucleic acid being produced by the target gene. This is done by creating chips with a specific set of base pairs (as opposed to sequencing chips, wherein every possible base pair combination is arrayed). Results are then compared to a reference or control, and the degree of change is noted. These chips are useful in diagnosing and treating diseases linked to particular genetic expressions, such as some forms of cancer.

The third type of chip is devoted to comparative genomic hybridization. It is designed to help clinicians determine the relative amount of a given genetic sequence in a particular patient. Using a healthy tissue sample as a reference and comparing it with a sample for instance from the diseased tumor usually does this.

It was demonstrated (PNAS USA. 1997, 94(4): 1119-1123) that controlled electric fields could be used to regulate transport, concentration, hybridization, and denaturation of single- and double-stranded oligonucleotides on DNA chips. Discrimination among oligonucleotide hybrids with widely varying binding strengths may be attained by simple adjustment of the electric field strength. When this approach is used, electric field denaturation control allows single base pair mismatch discrimination to be carried out rapidly (<15 sec) and with high resolution. Electric field denaturation takes place at temperatures well below the melting point of the hybrids, and it may constitute a novel mechanism of DNA denaturation.

Most currently available DNA chips are based on fluorescence detection technology that uses a laser to irradiate a sample and then measures the resulting fluorescence. Fluorescence detection methods commonly suffer from sensitivity barriers due to low signal to noise ratios, particularly with low concentration targets. Electrochemical detection allows for detection without the use of fluorescent (or other) labels and holds the potential for much higher sensitivity and shorter analysis time than currently available methodologies.

Electrochemistry has superior properties over the other existing measurement systems, because electrochemical biosensors can provide rapid, simple and low cost on-field detection. Electrochemical measurement protocols are also suitable for mass fabrication of miniaturized devices. Electrochemical detection of hybridization is mainly based on the differences in the electrochemical behavior of the labels towards the hybridization reaction on the electrode surface or in the solution.

Problems associated with the established fluorescence-based optical detection technique include the high equipment costs and the need to use sophisticated numerical algorithms to interpret the data. These problems generally limit its use to research laboratories and make it hard to adapt this detection scheme for on-site or point-of-care use. An electrical readout might be a solution to these problems. A review “Chip-based electrical detection of DNA” considers a number of different approaches to achieve an electrical readout for a DNA chip in IEE Proc. Nanobiotechnol., 2005, 152, 1.

A significant limitation of those dense arrays of oligonucleotides lies probably in the readout scheme. Fluorescent dyes are the standard label for gene chips. These dyes are expensive and they can rapidly photo bleach. Also the readout of those arrays involves highly precise and expensive instrumentation and needs sophisticated numerical algorithms to interpret the data, which makes the analysis time consuming. Because of these problems the fluorescence-based readout system is limited to research laboratories. For on-site and point-of-care applications analyzing systems are required that are cost efficient, fast, and easy to use. It is also not necessary to fit thousands of probes on one test, because there are often just a few well-defined parameters to be checked. Examples of such products include those on sale or soon to be marketed by Nanogen, Combimatrix and Toshiba.

Nanogen has been developing a technology allowing redistribution of DNA on the surface of the DNA chip and denature it electronically yet still requires fluorescence detection. The ability to apply a positive electric current to individual test sites on the microarray enables rapid movement and concentration of negatively charged DNA and RNA molecules and involves electronically addressing biotinylated samples, hybridizing complementary DNA reporter probes and applying stringency to remove unbound and nonspecifically bound strands after hybridization. It should be emphasized that all the movements of polynucleotides are happening in the boundaries of one DNA chip between the different parts of the chip. One or more test sites are activated with positive charge. Biotinylated samples or probes are bound to streptavidin permeation layer on the chip at those sites. Activated test sites are turned off, allowing for reporting. Red and green fluorescently labeled probes or samples are hybridized to bound complementary biotinylated strands. A system scans the chip and automatically analyzes red and green fluorescent ratios to determine results. After reporting, samples/probes are washed off and other samples can be added. Non-used (unactived, unbound) sites can be saved for future use. A single test site can be stripped and re-probed for multiple reporting. An aliquot from a single sample well can be bound to multiple test sites for high-level multiplex analysis.

Combimatrix uses the application of an electric potential to individual test sites on the microarray to synthesize oligonucleotides in situ on the DNA chip surface. Combimatrix currently markets fluorescence detection technology and has been developing electrochemical signal detection. This technology utilizes the redox enzyme amplification system. A DNA capture probe is synthesized at the electrode. The complementary target is a PCR product containing a biotin molecule that may be attached at the end of the sequence or to bases within the sequence. Streptavidin-labeled horseradish peroxidase is then added to the sample, and HRP binds to biotin on the DNA strand. Addition of substrate allows HRP to produce a product and a current at the electrode.

Toshiba has developed an electrochemical DNA chip for the single nucleotide polymorphism (SNP) typing of patients infected with hepatitis C. These chips are used to identify patients most likely to respond to interferon therapy. Capture probes are immobilized onto gold electrodes through a SAM. After the hybridization reaction to the target DNA, Hoechst 33258, an electrochemically active dye that specifically binds the minor groove of double-stranded DNA, is added. When an appropriate potential is applied, the oxidative current from the dye is proportional to the amount of bound target DNA.

Thus, there exists a need for a more efficient detection of a nucleic acid binding event in a DNA chip.

### SUMMARY OF THE INVENTION

A process for identifying a complementary nucleic acid probe to a target nucleic acid involves forming an array of spots where each spot of the array has an immobilized nucleic acid anti-probe to which is hybridized a nucleic acid probe to form a double-stranded anti-probe-nucleic acid probe complex. The array is placed in a solution filled array chamber and the anti-probe-probe complex is denatured. The nucleic acid probes are then moved within an electrophoretic field into a target chamber that includes a target nucleic acid. With multiple nucleic acid probes present within the target chamber, hybridization conditions are established to form double-stranded complexation between the target nucleic acid and nucleic acid probes in instances where the target nucleic acid has a sequence complementary to that of a nucleic acid probe. The nucleic acid probes noncomplementary to the target nucleic acid are then removed from the target chamber and allowed to rehybridize with the original anti-probes of the array or exposed to a series of immobilized anti-probes existing within a separate egress pathway. Determining whether the anti-probe spots exposed to nucleic acid probes noncomplementary to the target nucleic acid are single stranded after exposure to noncomplementary nucleic acid probes provides information as to target nucleic acid sequence. In an alternate embodiment, only nucleic acid probes complementary to target nucleic acids are exposed to immobilized anti-probes that are spatially isolated in spots either in the original array or within an egress pathway to determine comparable information. A return pathway is optionally provided to return some or all of the nucleic acid probes to the array so as to regenerate the array after testing.

An assemblage is provided for conducting such nucleic acid testing including at least an array chamber, a target chamber, and a nucleic acid probe permeable channel therebetween. Electrophoretic movement between the chambers is preferred. An egress pathway from the target chamber is optionally provided. Time of flight detection is also made possible by the inventive assemblage.

### BRIEF DESCRIPTION OF THE DRAWINGS

The present invention is further detailed with respect to the following nonlimiting figures. These figures depict only particular processes and apparatus according to the present invention with variants existing beyond those depicted.

FIG. 1A is a schematic of the concept of assembling of a nucleic acid microarray with external chamber providing space for hybridization of a nucleic acid target with a nucleic acid probe in solution;

FIG. 1B is a schematic denaturing of double-stranded oligonucleotides on the chip and electrophoretic transport of single-stranded DNA probes into the chamber with a nucleic acid target;

FIG. 1C is a schematic hybridizing a nucleic acid probe with target nucleic acids in solution;

FIG. 1D is a schematic transporting electrophoretically nucleic acid probes, which are not complementary to the target nucleic acid, back into a microarray chamber;

FIG. 1E is a schematic hybridizing free nucleic acid probes with DNA anti-probes of the same size immobilized on the microarray;

FIG. 2A is a schematic of an inventive assemblage of a DNA microarray with an external chamber for hybridization and with the labyrinth channel filled with solution having spots of immobilized DNA anti-probes on the bottom of the channel;

FIGS. 2B-2E are schematics of the operational steps for the assemblage of FIG. 2A where the steps so depicted are parallel to those of FIGS. 1B-1E, respectively;

FIG. 3 is a schematic depicting an operational mode for the assemblage of FIGS. 2A-2E in which a new array and new target nucleic acid are loaded into the respective chambers after an initial usage;

FIG. 4 is a schematic depicting an operational mode for the assemblage of FIGS. 2A-2E in which a new target nucleic acid is loaded into a target chamber and a recharged original array is provided after initial usage;

FIGS. 5A-5C are schematics of one mode of double-stranded nucleic acid denaturation through pH and electrode activation control;

FIG. 6A is a top view of a modular inventive assemblage well suited for manufacture to perform a process as depicted in FIGS. 1A-1E;

FIG. 6B is a central cross section through the assemblage of FIG. 6A;

FIGS. 7A-7E are cross-sectional schematics of a process of operating the assemblage of FIGS. 6A and 6B. FIG. 7A: manufacturer preloading of chambers with solutions, FIG. 7B: loading target nucleic acid sample into target chamber, FIG. 7C: electrophoretically transporting and hybridizing nucleic acid probes with target nucleic acid, FIG. 7D: electrophoretically returning nucleic acid probes not complementary to the target nucleic acid to the array chamber, FIG. 7E: hybridizing nucleic acid probes not complementary to the target nucleic acid to anti-probes immobilized and spotted in the array, and FIG. 7F: determination of double strand complex in a given spot by flow of washing and dye solutions;

FIG. 8 is a schematic of an alternate embodiment of an inventive assemblage having a target nucleic acid chamber in fluid communication with multiple probe-anti-probe arrays;

FIG. 9 are schematics depicting the connection of various inventive microarrays with chambers for hybridization in solution and with a channel for transporting nucleic acid probes by electrical field and detecting the probes passing through a detector;

FIG. 10 is a schematic depicting the two microarrays with a chamber for DNA hybridization in solution and with a channel for transporting nucleic acid probes by electrical field and detecting the probes passing through the detector;

FIG. 11 is a schematic depicting the connections of three DNA microarrays with a chamber for DNA hybridization in solution and with a channel for transporting along DNA probes by electrical field and detecting said DNA probes passing through the detector.

### DETAILED DESCRIPTION OF THE PREFERRED EMBODIMENTS

The present invention has utility as a process and assemblage for identifying complementary sequences between a target nucleic acid and an array of nucleic acid probes initially forming a complex with an immobilized anti-probe nucleic acid sequence. A new process for signal detection on a DNA chip is provided in which the flow of charged nucleic acid probes released from anti-probe spots is determined by a detector as part of an inventive assemblage or an appended labyrinth based on the probe path and/or time of fight to a detector. According to the present invention, in addition to nucleic acid probes which are immobilized on a DNA microarray and nucleic acid targets which according to the prior art are free DNA molecules in solution, the present invention introduces an array of immobilized anti-probes of known sequence. The probes are selectively denatured from the anti-probes and brought into contact with a target nucleic acid under conditions in which hybridization between a nucleic acid probe and the target nucleic acid can occur of the probe nucleic acid sequence is complementary to that of the target nucleic acid. Thereafter, moving noncomplementary probes into contact with a series of immobilized complementary anti-probes under hybridization conditions, detection of those mobilized complementary anti-probes by various means that are not present as double-stranded complexes with nucleic acid probes indicates if a nucleic acid probe is complementary to the target nucleic acid. By separation of the target nucleic acid from the array of probe-anti-probes with a gel providing nucleic acid probe communication through electrophoretic movement, the target nucleic acid is provided in a variety of forms illustratively including free molecules in solution, as is known in the prior art, as well as immobilized on a solid surface, embedded in porous media such as a gel, adhered to particulate which is paramagnetic particles, semiconductor particles, metal particles or the like.

The present invention trough the inclusion of immobilized anti-probes capable of selectively being hybridized to nucleic acid probes that are amenable to transport affords the user multiple modes of operation with the resultant advantages illustratively including regeneration of the probe-anti-probe array, high throughput detection, time of flight detection, and combinations thereof. As a result, the present invention is amenable to a high level of manufacture so as to increase user throughput and provide target nucleic information consistent with that obtained from various types of prior art DNA chips. These uses illustratively include high throughput genotyping, resequencing, single nucleotide (SNP) genotyping, and gene expression chips. As a result, the present invention offers a degree of flexibility in operation, simplified manufacture and operation, and in regard to certain embodiments allows one to regenerate the inventive array for subsequent usage.

According to the present invention it is possible to prepare a complex of anti-probes and nucleic acid probe by first preparing a long double stranded nucleic acid which after treatment with specific restriction enzymes the second strand becomes a number of short nucleic acid strands hybridized to an elongated anti-probe strand. This procedure facilitates manufacture of numerous copies of nucleic acid probes by first amplifying long and repetitive double strand nucleic acid molecules and then treating such long double strand nucleic acid molecules with the appropriate restriction enzymes.

The present invention relies on a carrier capable of uniquely and reversibly binding a nucleic acid probe. In an inventive array, anti-probes are preferably isolated dimensionally in space or on a substrate. It is appreciated that in an array according to the present invention with anti-probes immobilized on a surface or within a porous matrix, nucleic acid probes can be harvested from a random mixture of short oligonucleotides, having a length of between 5 and 50 bases. Oligonucleotides harvested from the random mixture can be used as nucleic acid probes for subsequent hybridization and use in assays.

The arrangement of anti-probes in space so as to provide an inventive array includes a number of options in manufacture and operation. By way of example, anti-probes are coupled together to form an elongated strand. Preferably, the identity and position of each anti-probes along the strand is known. More preferably, spacer segments are provided intermediate between anti-probes along a strand so as to disfavor steric hindrance with probes pairing with the anti-probes sequences along the strand. It is appreciated that the specific inclusion of restriction sites within linker segments of the strand or knowledge as to such sites within anti-probes nucleic acid sequences provides for subsequent modification to replace a given anti-probes with a new anti-probe having different specificity. The ability to produce an elongated strand of anti-probes secured to a substrate by one or more strand termini creates an interaction environment with a probe in solution that is largely free of substrate surface interaction and the hindrances to probe-anti-probe complexation associated with a monolayer of probes immobilized on a substrate spot as in a conventional DNA microarray.

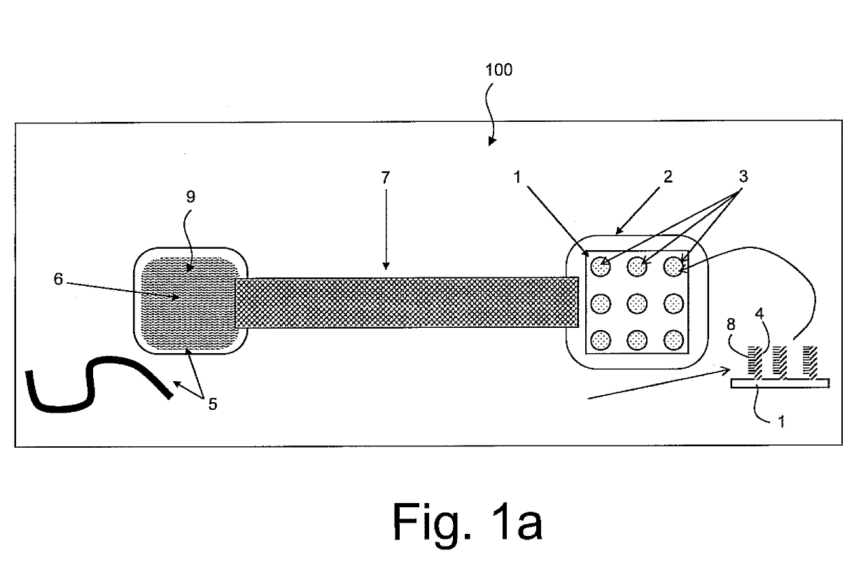

The operation of an inventive assemblage is illustrated in FIGS. 1A-1F. Throughout FIG. 1 like numerals are used throughout to depict the movement and status of various components throughout the sequence of an inventive process. An array of immobilized anti-probe spots 4 are provided on an array substrate 1. An anti-probe 4 is a nucleic acid having a sequence complementary to a single-strand oligonucleotide used herein as a nucleic acid probe 8. The anti-probe is readily immobilized on the substrate 1 through conventional techniques illustratively including covalent attachment to a functional group on the solid surface or biotinylation. It is appreciated that while an anti-probe 4 in simplest form includes only a single sequence complementary to a nucleic acid probe 8, an anti-probe having repeated sequences along the length thereof allows one to decrease anti-probe density within a spot, increase the number of nucleic acid probes 8 that can be hybridized to a spot, or a combination thereof. An anti-probe formed as a strand having multiple sequences complementary to nucleic acid probes is amenable to securement to a substrate 1 in an area to define a spot at one end or at both ends of the strand. After immobilizing anti-probes 4 onto substrate 1, the anti-probes are hybridized to complementary nucleic acid probes. Preferably, the anti-probes in a given spot 3 are known and vary in sequence relative to other spots on the array 1. Nucleic acid probes 8 complementary to nucleic acid anti-probe 4 are readily collected from a random mixture of short nucleic acid oligonucleotides or alternatively selected from a library. With the introduction of a solution of nucleic acid probes 8 into contact with array substrate 1 having immobilized anti-probes thereon under hybridization conditions, a double-stranded complex of anti-probe oligonucleotide with a complementary nucleic acid probe is formed in the multiple spots 3. It is appreciated that the anti-probe 4, in addition to being immobilized on a surface of array substrate 1, is readily isolated in a solution volume by porous media that is exclusive of the anti-probe in strand form while being permissive to nucleic acid probe movement. Alternatively, an anti-probe strand is isolated in a gel that is permissive to nucleic acid probe transport.

With the formation of double-strand complex of immobilized anti-probe 4 and free nucleic acid probe 8 to form spots 3 on array substrate 1, the array substrate 1 is placed in an array chamber 2. The array chamber 2 is in fluid communication with a target nucleic acid chamber 6 containing a target nucleic acid 5. In the lower left and right corners of each of FIGS. 1A-1F, cross-sectional, nonscaled images of the strandedness of the anti-probe 4 on substrate 1, and target nucleic acid 5, respectively, are provided.

The array chamber 2 and target chamber 6 are flooded with electrophoretic buffer solution and brought into fluid communication by way of a channel 7 through which nucleic acid probes 8 are amenable to transport while the target nucleic acid 5 is excluded from transport or at least travels through the channel 7 at a rate of less than 10% of the rate of nucleic acid probe movement. As used herein, a nucleic acid probe typically has a length of between 5 and 60 single-strand bases, and preferably between 5 and 50 bases. In contrast to the short nucleic acid probe oligonucleotides, a target nucleic acid 5 typically has a length of greater than 200 single-strand bases. It is appreciated that a target nucleic acid is present within target chamber 6 in a variety of forms. These forms include free-floating nucleic acid, as is conventional to the art; adhered to a surface of a substrate 9; attached to a particle such as a paramagnetic particle, a metal particle, semiconductor particle, or polymeric bead; or trapped within a volume by a size exclusive porous media permissible to nucleic acid probes; or trapped within a gel. As a result, it is appreciated that a DNA chip having target nucleic acid sequences adhered to a surface of a substrate 9 is operative with an inventive assay assemblage.

While the nature of the media within channel 7 can include size exclusive membranes, chromatographic media or gels, in a preferred embodiment the media within channel 7 is a gel amenable to electrophoresis. It is appreciated that various gels are now commonly used for a nucleic acid electrophoresis, these gels illustratively including polyacrylamide and agarose.

After forming the assay assemblage of FIG. 1A, electrophoretic electrodes 10 and 12 are introduced into the array chamber 2 and target chamber 6, respectively. The electrodes 10 and 12 are then coupled to an electrophoretic power supply 14. After denaturing the double-stranded anti-probe oligonucleotide-nucleic acid probe complexes arrayed in spots 3, the power supply 14 is energized to move the nucleic acid probes 8 through the channel 7. Demobilized anti-probes 4 remain adhered to substrate 1. Electrophoresis occurs until the nucleic acid probes 8 reach the target chamber 6 and the ability to interact with target nucleic acid 5.

Throughout FIG. 1 like numerals are used throughout to depict the movement and status of various components throughout the sequence of an inventive process.

The array of double-stranded complex spots 3 after denaturation and transport of nucleic acid probes 8 are now single-stranded anti-probe 4 remaining in position and denoted as white spots at 15 in FIG. 1B.

After establishing hybridization conditions within the target chamber to form a target nucleic acid-nucleic acid probe double-stranded complex, some nucleic acid probes 8 are hybridized to target nucleic acid 5 while other probes remain single stranded and in solution. Upon again establishing an electrophoretic potential between array chamber 2 and test chamber 6 with a reversed polarity relative to that depicted in FIG. 1B, those nucleic acid probes 8 which are not complementary in sequence to the target nucleic acid 5 remain single stranded in solution. These single-stranded free nucleic acid probes then migrate under the influence of the electric field to return to the assay chamber 2. After establishing hybridization conditions within the assay chamber 2, double-stranded anti-probe-nucleic acid probe complexes are formed only in those spots where the nucleic acid probe was not complementary for a sequence of the target nucleic acid 5. Subsequent to establishing hybridization conditions within assay chamber 2, a mixture of double-strand spots 3 and single-strand spots 15 exists on the substrate 1. The detection as to which nucleic acid probes derived from the array substrate 1 which are complexed to target nucleic acid 5 are identified by a number of methodologies conventional to the DNA chip art that involve fluorescent or other spectroscopic interrogation of target DNA. Preferably, an inventive assemblage according to FIG. 1 is developed to determine if the nucleic acid probe associated with a given spot is present on the substrate 1 by introducing a dye species that distinguishes between single-strand and double-strand compositions within each of the spots with knowledge as to the specific sequence of each anti-probe spotted on the array of substrate 1, as shown in FIG. 1E.

It is appreciated that the array of substrate 1 as depicted in FIG. 1A is regenerated to the original state after determination of homology between target nucleic acid and anti-probe according to FIG. 1E by denaturing the target nucleic acid double-stranded complex with complementary nucleic acid probes 8 and then repeating the electrophoretic migration of FIG. 1D and the nucleic acid probe-anti-probe hybridization of FIG. 1E followed by washing to remove single-stranded structure resolving dye present within the assay chamber 2.

An alternative determination of complementary nucleic acid probe identity to target nucleic acid is provided by the inclusion of a separate nucleic acid probe egress pathway. An inventive assemblage containing an egress pathway is particularly well suited for instances where the identity of anti-probe sequences is unknown, detection techniques other than through the inclusion of a dye is desired, subsequent chemistry is to be performed on the nucleic acid probes, or a combination thereof.

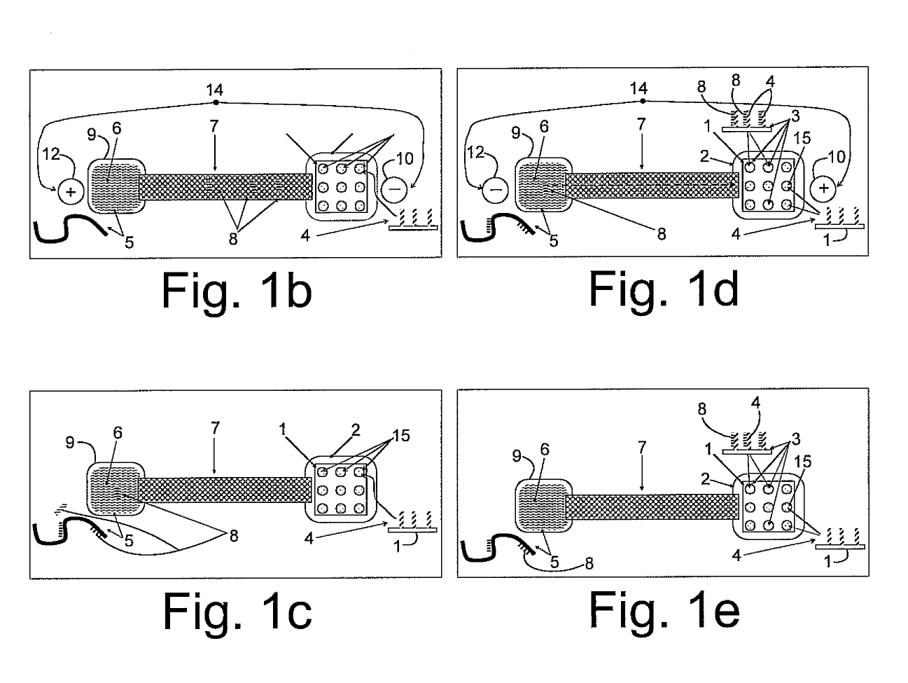

An inventive assemblage inclusive of an egress pathway is depicted in FIGS. 2A-2E where like numerals correspond to those detailed above with respect to FIG. 1. The egress pathway 18 depicted in FIGS. 2A-2E is provided as a blank form labyrinth. It is appreciated that numerous other pathways are operative in formation of an egress pathway. These forms illustratively include linear, arcuate, acute angular, and combinations thereof.

Referring now to FIG. 2A, an inventive assemblage 100 as detailed with respect to FIG. 1A is provided along with the inclusion of an egress pathway 18. The egress pathway 18 is filled with electrophoretic buffer solution and has a series of spots 20 of immobilized anti-probe 22 immobilized onto a surface 24 of the pathway 18. A cross-sectional schematic with elements not to scale is provided in the upper left portion of FIGS. 2A-2E depicting the strandedness of the immobilized anti-probes 22. The anti-probes 22, like anti-probes 4, are also readily in a solution well excluding anti-probe movement by a porous media, or embedding within a gel such that the porous media or gel are permeable to nucleic acid probes coming in contact therewith.

FIG. 2B depicts the denaturation of the double-stranded complex between anti-probe 4 and nucleic acid probe 8 and the migration of nucleic acid probes within an electric field as detailed above with respect to FIG. 1B.

After allowing nucleic acid probes to enter a target chamber 6 and interact with target nucleic acid 5 under hybridization conditions, nucleic acid probes 8 complementary to target nucleic acid 5 form a double-stranded target-probe complex as shown in FIG. 2C and uncomplementary, single-strand nucleic acid probes 8 are then moved into the egress pathway 18 under the influence of an electric potential established between electrode 10 positioned within the array chamber 2 and electrode 12 positioned at the terminus 26 of the pathway 18. The power supply 14 supplies a potential between electrodes 10 and 12. A single-stranded nucleic acid probe 8 traveling along pathway 18 hybridizes to a complementary anti-probe 22 to form a double-stranded pathway anti-probe-nucleic acid probe double-stranded complex 22-8 by converting a single-stranded spot 20 into a double-stranded spot denoted at 30.

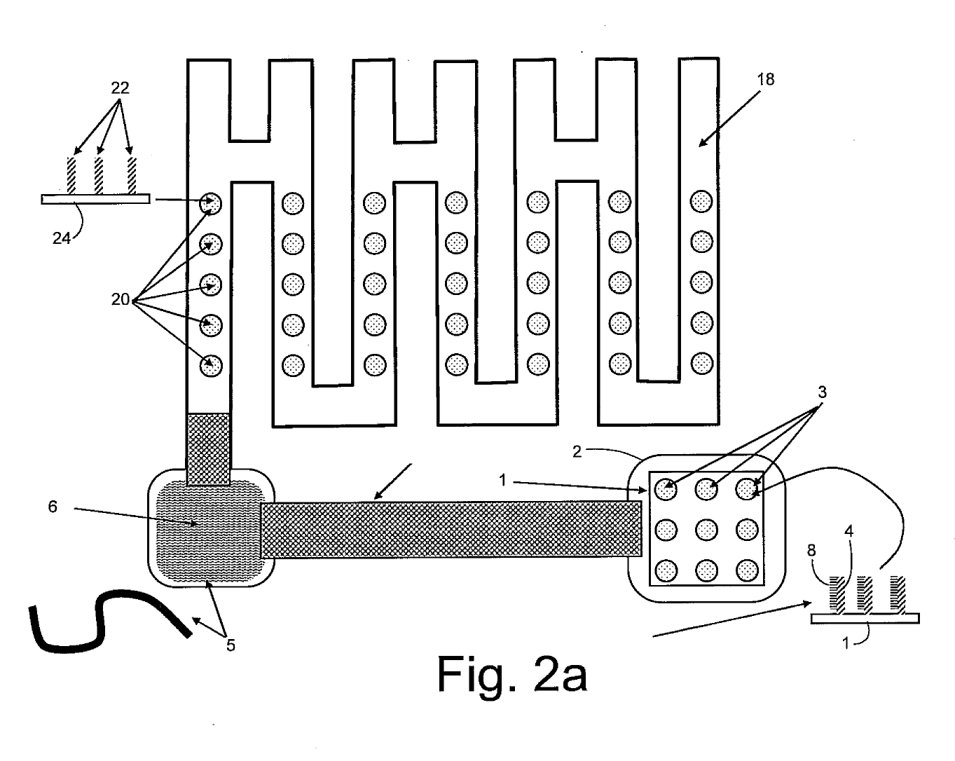

The detection as to whether a given spot is a single-stranded anti-probe 20 or a double-stranded complex spot 30 again is amenable to conventional dye techniques such as the inclusion of a dye selective for either a single-strand or double-strand structure is spatially resolve the nature of each spot. Preferably, the resolution of spots as to single-strand spots 20 or double-stranded complex spots 30 involves time of flight from a spot to a detector 32. Since the distance between a given spot 20 or 30 and a detector 32 is known, the spacing between successive spots is known, and the molecular weight of a nucleic acid probe, time of flight detection as to the strandedness of a given spot is readily performed. In the embodiment depicted in FIG. 2D, multiple electrophoretic paths are created that encompass within that path a nucleic acid probe detector. The detector 32 functions based on spectrophotometrics or a change in electrical signal associated with a nucleic acid probe movement past a detector sensing a property such as conductivity or an electrophoretic voltage change necessary to maintain total power supplied across the electrodes. FIG. 2E shows the detector output for each of the detector lines 1-7 depicted in FIG. 2D in which a solid line denotes the absence of a nucleic acid probe while the dashed line denotes the detection of a nucleic acid probe that previously was part of complex 28. The eliciting was a function of time that corresponds to the series of five spots potentially feeding signal to each of lines 1-7. It is appreciated that any effluent from egress path 18 including that leaving terminus 26 depicted in FIG. 2C or from any one of lines 1-7 is readily returned to array chamber 2 to allow nucleic acid probes noncomplementary to the target nucleic acid to rehybridize to the anti-probes 4. Additionally, the complex between nucleic acid probes and target nucleic acid within chamber 6 is readily denatured from the complementary nucleic acid probes returned to array chamber 2 by reversing the polarity of the electrophoresis between electrodes 10 and 12 relative to FIG. 2B. Subsequent to rehybridization of the complementary nucleic acid probes to respective anti-probes 4, the assemblage is returned to the status depicted in FIG. 2A. Following completion of a test and the decision not to return nucleic acid probes to array chamber 2, the spent array substrate 1 is removed along with the test chamber 6. A new array 1′ and new nucleic acid target 6′ are placed in the respective chambers and a subsequent test then performed. This mode of operation is depicted schematically in FIG. 3.

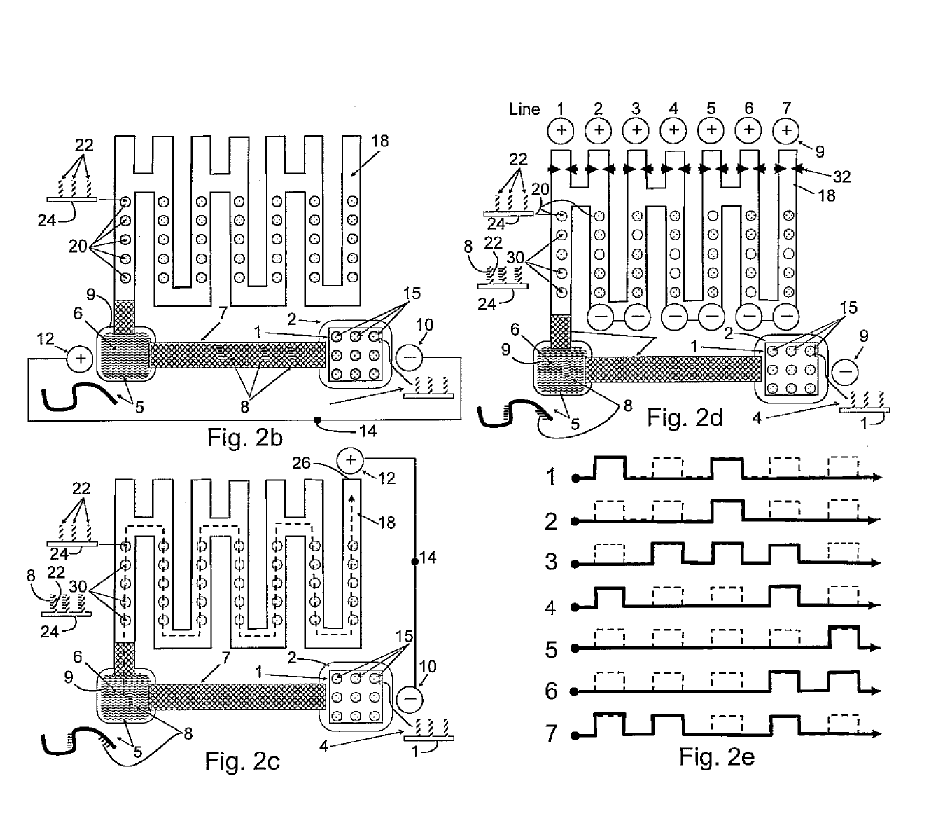

In an alternative embodiment, subsequent to hybridization between the target nucleic acid 5 and complementary nucleic acid probes 8 to form a double-strand complex, the noncomplementary nucleic acid probes are returned via channel 7 to array chamber 2 and thereafter the double-stranded complex between complementary nucleic acid probes and target nucleic acid 5 are denatured with the complementary nucleic acid probes entering egress pathway 18 to produce an opposite spot pattern relative to that depicted in FIGS. 2C and 2D as well as the opposite line outputs of FIG. 2E. In this operational mode, effluent containing target nucleic acid complementary probes are also optionally recycled to the array chamber 2 such that following rehybridization the array substrate 1 is returned to an original state. Cycling of an array is appreciated to be of considerable value in high throughput automated testing. This operational scheme is depicted schematically in FIG. 4 with the recharging of the original array, removal of target chamber 6 and performing a new test with a new target chamber 6′ containing a different or potentially different target nucleic acid.

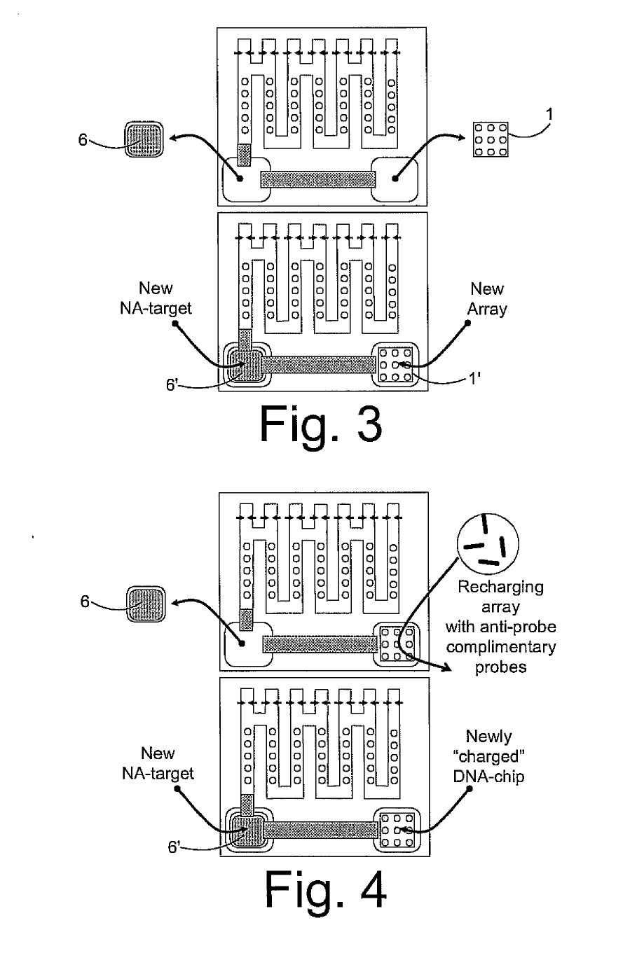

While there are numerous techniques known to the art for denaturing a double-strand nucleic acid complex, illustratively including heating, changes in pH, changes in ionic strength, and combinations thereof, an additional mode of inducing complex denaturation is detailed with respect to FIGS. 5A-5C. A neutral pH solution with both electrodes turned off represents a default state as shown in FIG. 5A, top panel. When both electrophoretic electrodes are activated, a sphere of low pH develops proximal to the electrodes as shown in FIG. 5A, lower panel. The electrolytic buffer solution of a high pH electrode activation creates a neutral pH region proximal to activated electrodes as shown in FIG. 5B, top panel. As a result, through adjustment of the buffer solution pH and switching electrodes between energized and deactive states allows a pH only in a vicinity of one electrode and double-stranded nucleic acid complex denaturation thereby releasing nucleic acid probe species from the spot in question, as shown in FIG. 5B, lower panel. Through the movement of a positive electrode downstream from a detector, free nucleic acid probe species migrate past a detector at a time indicative of the spot denoted in FIG. 5C as “electrode turned off.”

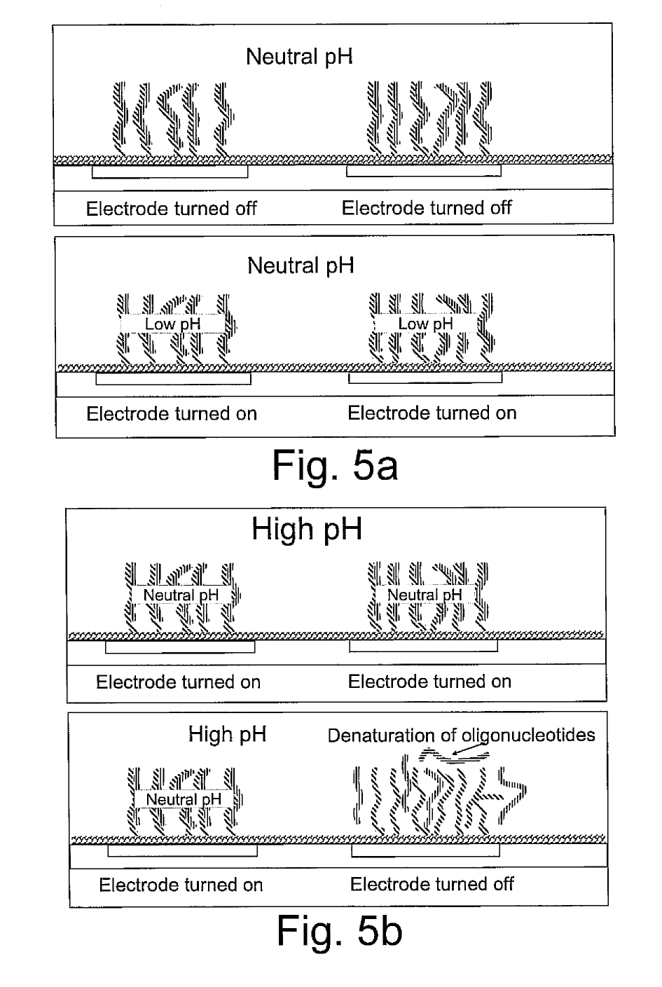

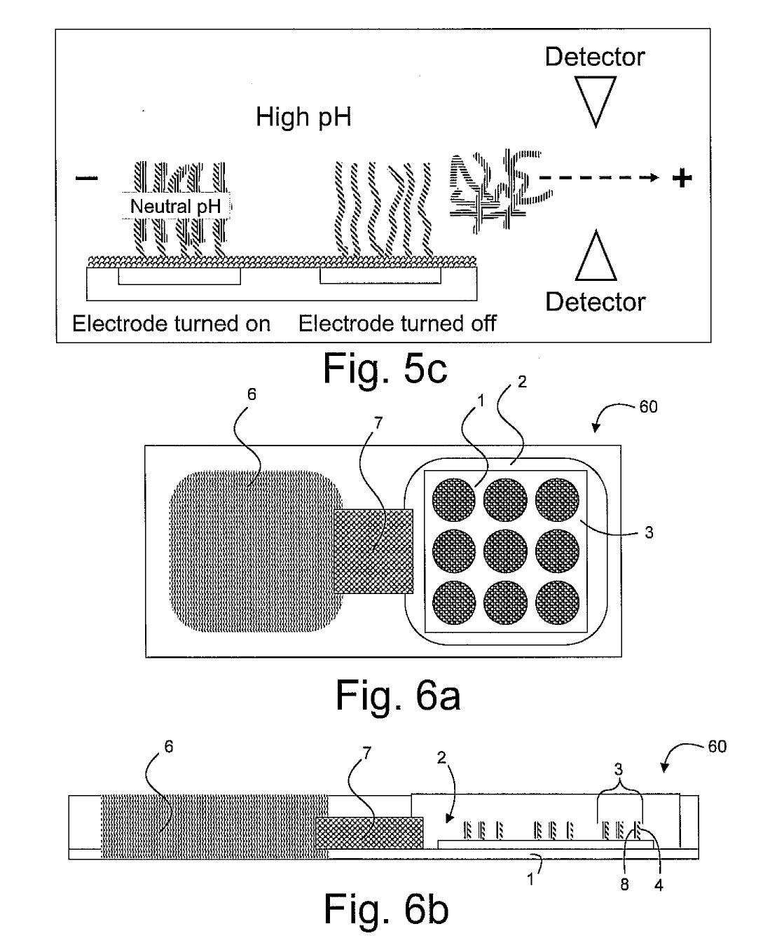

Top and cross-sectional views of a modular inventive assemblage well suited for manufacture to perform a process as depicted in FIGS. 1A-1E is shown generally at 60, where like numerals correspond to those used with respect to the preceding figures. The FIGS. 7A-7E are cross-sectional schematics of a process of operating the assemblage of FIGS. 6A and 6B, where lice numerals correspond to those used with respect to the preceding figures. In FIG. 7A, the manufacturer preloads of chambers with solutions. In FIG. 7B, a target nucleic acid sample is loaded into the target chamber. Thereafter, nucleic acid probes are electrophoretically transported and hybridized with target nucleic acid to which probe is complementary, as shown in FIG. 7C. Nucleic acid probes not complementary to the target nucleic acid are electrophoretically returned to the array chamber, as shown in FIG. 7D. Hybridization of those nucleic acid probes not complementary to the target nucleic acid to anti-probes immobilized and spotted in the array provides the identity of the probes sequences complementary to the target nucleic acid through imaging difference between single stranded anti-probes and double stranded probe-anti-probe complexes, as shown in FIG. 7E. The flow of washing and dye solutions through a conduit 62 allows one to determine strandedness in a given spot without opening the modular inventive assemblage 60.

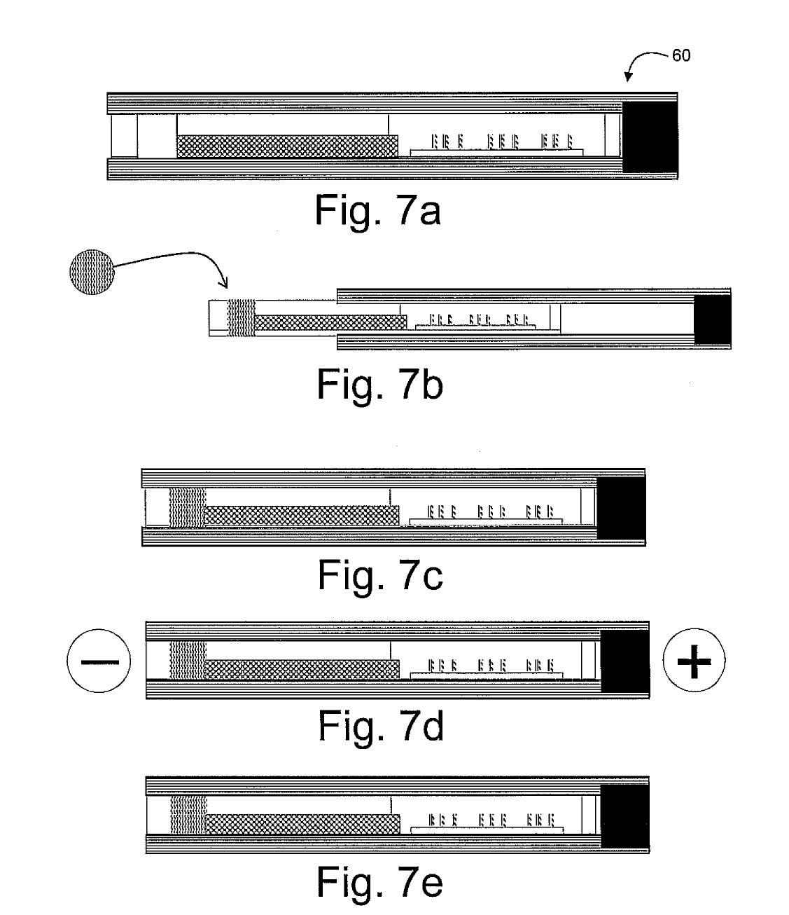

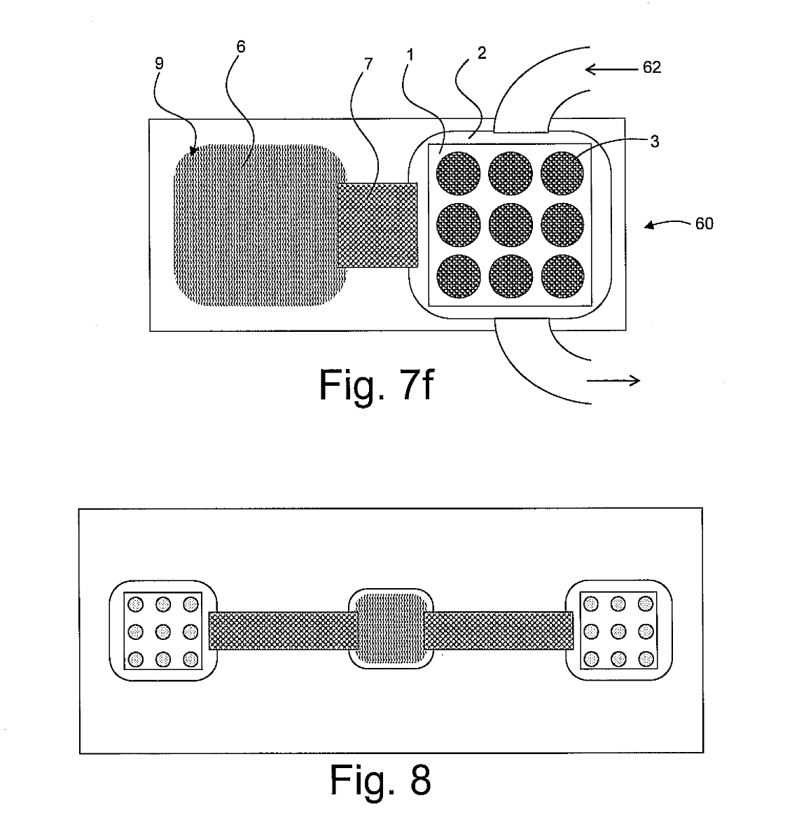

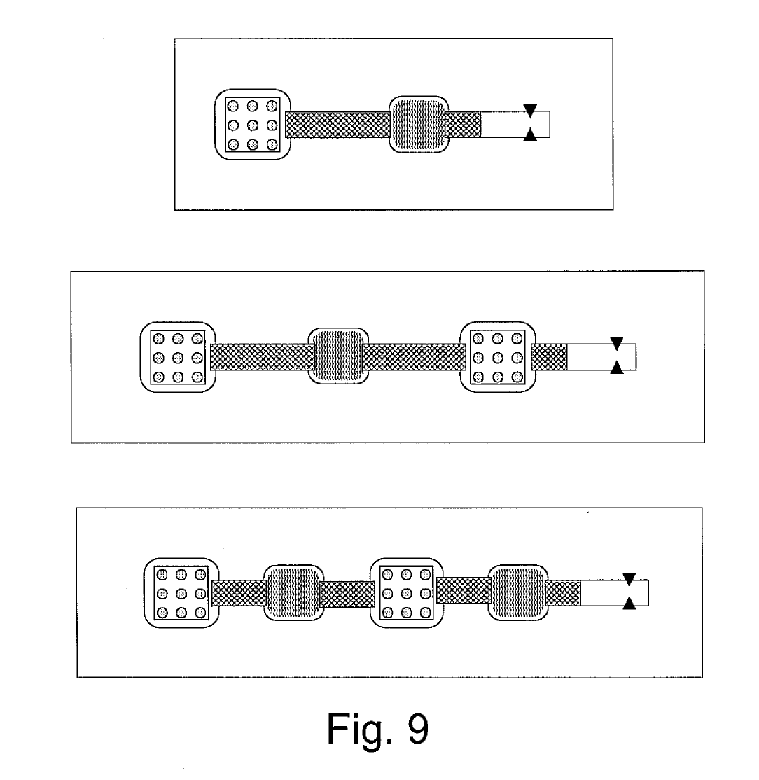

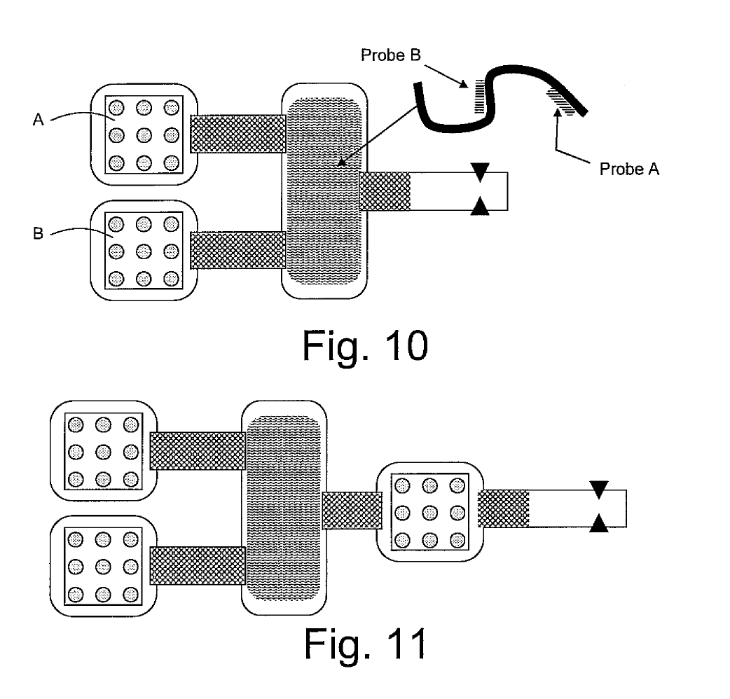

In addition to the embodiments of the inventive assemblage depicted in FIGS. 1 and 2 in which a single array of probes bound to immobilized anti-probes interacts with a single test chamber and optionally includes an egress pathway, it is appreciated that the inventive concepts are readily extended to various combinations of probe arrays, target chambers, and detectors as depicted in FIGS. 8-11. While the embodiments depicted in FIGS. 9-11 include a detector at the rightmost extreme of the assemblage, it is appreciated that these embodiments as well as those depicted in FIG. 8 are readily modified to include one or more egress channels as detailed with respect to FIG. 2. Optionally, any number of lines as depicted in FIG. 2D are also provided to an egress pathway to facilitate alternate modes of detection.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A process for identifying a complementary nucleic acid probe to a target nucleic acid comprising:
forming an array of spots, each spot comprising a nucleic acid probe, a nucleic acid probe hybridized to a respective immobilized oligonucleotide anti-probe to yield a double-stranded anti-probe-nucleic acid probe complex;
placing said array in a solution filled array chamber;
denaturing said double-stranded oligonucleotide anti-probe-nucleic acid probe complex;
moving said nucleic acid probe electrophoretically into a target chamber comprising a target nucleic acid;
establishing hybridization conditions in said target chamber to form a target nucleic acid-nucleic acid probe double-stranded complex when the target nucleic acid has a complementary sequence to said nucleic acid probe;
transporting a nucleic acid probe noncomplementary to the target nucleic acid into contact with a series of immobilized anti-probes;
hybridizing each of said nucleic acid probes noncomplementary to the target nucleic acid to one of said series of immobilized anti-probes; and
determining whether each of said series of immobilized anti-probes exist as present as a single strand.

2. The process of claim 1 wherein said anti-probe is a strand.

3. The process of claim 1 wherein moving said nucleic acid probe electrophoretically into the target chamber occurs through a gel.

4. The process of claim 1 wherein the target nucleic acid within said target chamber is untethered.

5. The process of claim 1 wherein the target nucleic acid within said target chamber is bound to a particle.

6. The process of claim 5 wherein said particle is paramagnetic.

7. The process of claim 1 wherein the target nucleic acid within said target chamber is embedded within gel.

8. The process of claim 1 wherein the target nucleic acid within said target chamber is adhered.

9. The process of claim 1 wherein said series of immobilized anti-probes include said anti-probe within said array of spots.

10. The process of claim 1 wherein said series of immobilized anti-probes extend from an egress pathway in fluid communication with said target chamber.

11. The process of claim 10 wherein determining whether one of said series of immobilized anti-probes exists in single-strand form as determined by time of flight between each spot and a detector.

12. The process of claim 1 further comprising denaturing said target nucleic acid-nucleic acid probe double-stranded complex and returning said nucleic acid probe to which said target nucleic acid has the complementary sequence to said array chamber, and rehybridizing said array of spots to return each spot of said array of spots to the form of the double-stranded oligonucleotide anti-probe-nucleic acid probe complex.

13. The process of claim 10 further comprising recycling effluent from said egress pathway to said array of spots.

14. The process of claim 1 further comprising exposing under hybridization conditions the target nucleic acid to a second series of nucleic acid probes, said second series of nucleic acid probes originating from a second array of spots, each spot of said second array of spots comprising a second nucleic acid probe hybridized to a respective second immobilized nucleic acid anti-probe.

15. The process of claim 1 further comprising exposing said nucleic acid from said array of spots to a second target chamber comprising a second target nucleic acid.

16. The process of claim 1 wherein determining whether each spot of said series of immobilized complementary anti-probes is single stranded comprises:
creating a high pH solution environment;
deactivating an electrode proximal to each spot of said series of immobilized complementary anti-probes to denature any double-stranded complex associated with each spot; and
detecting the passage of nucleic acid probe as a function of time of flight.

17. A nucleic acid assay assemblage comprising:
an array chamber containing nucleic acid probes each immobilized to a complementary nucleic acid anti-probe in the form of a double-stranded complex;
a target chamber containing a target nucleic acid;
a channel permeable to said nucleic acid probes in fluid communication between said array chamber and said target chamber; and
a fixture for coupling an electrophoretic electrode to said assay chamber and a second electrophoretic electrode to said target chamber.

18. The assemblage of claim 17 wherein the channel comprises a gel permeable to said nucleic acid probes.

19. The assemblage of claim 17 further comprising an egress pathway in fluid communication with said target chamber.

20. The assemblage of claim 17 further comprising a detector operating on time of flight.

21. The assemblage of claim 19 wherein said egress pathway further comprises multiple electrophoretic electrodes along a pathway length.

22. The assemblage of claim 19 further comprising a return pathway between said egress pathway and said array chamber independent of said target chamber.
